(19) United States
(12) Patent Application Publication
(10) Pub. No.: US 2024/0133368 A1
(43) Pub. Date: Apr. 25, 2024

(54) Shape Memory Alloy Capsule Micropump for Drug Delivery Applications

(71) Applicant: The American University in Cairo, New York, NY (US)

(72) Inventors: Youssef Mohamed Kotb, New Cairo (EG); Islam Adel Elgamal, New Cairo (EG); Mohamed Yousef Serry, New Cairo (EG)

(21) Appl. No.: 18/275,672

(22) PCT Filed: Mar. 29, 2022
(86) PCT No.: PCT/US2022/022517

§ 371 Date: Aug. 2, 2023

Related U.S. Application Data
(60) Provisional application No. 63/167,783, filed on Mar. 30, 2021.

(51) Int. Cl. F03G 7/06; A61M 31/00; F04B 19/00

(52) U.S. Cl. CPC F03G 7/0646; A61M 31/002; F03G 7/0614; F04B 19/006; A61M 2205/0266

(57) ABSTRACT

An SMA actuated capsule micropump is provided having a linear actuation which leads to large deflection, high discharge volume, and high static head pressure. The pump is designed for drug delivery applications by introducing a replaceable capsule reservoir and controlling the drug's delivery at a constant dose and a constant static head pressure. The device has a wide range of flow rates (from less than 2 to more than 2500 μL/min) that would be suitable for a broad range of drug delivery applications. Also, the pump has a wide range of pressures up to 14 kPa (105 mmHg) that are possible, which well exceeds the back pressure of most of the superficial veins typically utilized for intravenous drug delivery, without compromising the suitability for transdermal drug delivery.

### FIELD OF THE INVENTION

The invention relates to drug delivery devices and methods. In particular, the invention relates to shape memory alloy capsule micropumps.

### BACKGROUND OF THE INVENTION

Implantable drug delivery systems are gaining much attention in recent years due to their advantages over conventional drug delivery methods (e.g. oral and intravenous). They allow the use of higher localized concentrations, thus making the treatment more effective by delivering the drug directly to the tissue. Lessening the spread through the unintended body tissues, and controlling drug release can be optimized for maximum efficiency and a minimum amount of drug used, thus decreasing the cost of treatment. This also enables drug delivery to immunologically isolated parts of the body (e.g. cornea), unreachable with conventional drug delivery.

Although controlled passive-release drug delivery devices such as microencapsulation, bio-adhesives, polymer implants, and transdermal patches are currently the most dominant drug delivery methods; they are designed to deliver small amounts of the drug gradually and at a constant rate through difference in concentration between the drug and the surrounding tissue, but not capable of delivering the drug in a nonlinear fashion, i.e. they are nonprogrammable and cannot deliver the drug on-demand. Moreover, some clinical situations (e.g. delivery of hormones, anticancer agents, and vaccines) necessitate either external control and configurability of the drug delivery rate or dose beyond the existing controlled passive-release drug delivery capabilities methods.

Accordingly, there has been an increased interest in miniature active-controlled drug delivery systems utilizing wearable or implantable micropumps capable of delivering microliters of medicine on demand by forcing the delivery through pressure difference rather than the concentration difference between drug and the surrounding tissue.

Smart material actuators have gained enormous appeal for manufacturing wearable and implantable micropumps due to their high energy density. However, smart material actuators generally have several shortcomings involving either stroke or pressure limitations.

Shape memory alloy (SMA) actuators are based on NiTi metallic alloys that can recover permanent strain by changes either in stress, temperature, or a combination of both. SMA generates large strain when temperature changes due to diffusion-less transformation (twinning/detwinning) from martensite to austenite phases.

There are several advantages to SMA-actuated micropumps, including (i) being a direct drive actuator with solid-to-solid phase transformation (i.e. absence of friction, no moving or reductions mechanisms required, and noiseless), which also leads to the device being compact and more reliable and more suitable for miniaturized systems, (ii) large strokes can be realized, (iii) operating quasi-statically and independent of patient motion or body orientation, (iv) can provide high power-density; thus high flow rates and high pressures can be attained with miniaturized pumps, (v) excellent corrosion resistance and biocompatibility.

On the other hand, the main disadvantage with SMA micropumps is their low actuation frequency since the phase change in SMA is achieved by heat exchange with the surroundings heat source or a heat sink. Thus, the heating and cooling rates determine the frequency response of the SMA pump.

Several thin-film SMA strip and diaphragm type micropumps with high reciprocation rates have been widely reported. One thin-film SMA diaphragm pump is capable of pumping up to 50 μL/min. A reciprocating SMA peristaltic pump designs which can pump up to 1000 μL/min of fluid were also reported. However, both the diaphragm and peristaltic designs used in SMA micropumps do not allow large displacements, thus limiting the pump's discharge volume, throughput, and static head pressure.

One application of SMA micropump is for chemical delivery to biochemical integrated circuit chips. However, the use of SMA micropumps for implantable drug delivery applications necessitates online configurability, operating with no vibrations and at a low energy budget (i.e. battery-powered) and being small in size.

Therefore, in this invention, the inventors present a new design of a high-power density resistively actuated SMA capsule micropump for drug delivery applications.

### SUMMARY OF THE INVENTION

A shape memory alloy (SMA) actuated micropump is provided and useful for drug delivery applications. The design integrates a built-in replaceable drug reservoir within the pump package forming a self-contained preloaded capsule pump with an overall pump volume of, in one example, 424.7 μL. The design results in a compact, simple, and inexpensive micropump and reduces the probability of contamination with attained almost zero dead volume values. The pump has NiTi-alloy SMA wires coiled on a flexible elastomeric enclosure and actuated by joule heating. Unlike diaphragm and peristaltic SMA micropump designs that actuate transversely, the design is actuated longitudinally along the direction of the highest mechanical compliance resulting in large strokes in the order of 5.6 mm at 27% deflection ratio, actuation speed up to 11 mm/s, and static head pressures up to 14 kPa (105 mmHg) at 7.1 W input power; thus, high throughputs exceeding 2524 μL/min under free convention conditions could be achieved. Through modeling the inventors found that a low stiffness enclosure material combined with thinner SMA wire diameter would result in the maximum deflection at the lowest power rating. To prove its viability for drug delivery applications, the pump was operated at a constant discharge volume at a relatively constant static head pressure. Furthermore, a design of bicuspid-inspired check-valves is presented and integrated onto the pump to regulate the flow. Since the built-in reservoir is replaceable, the pump capsule can be reused multiple times and for multiple drug types.

In one embodiment, the invention is a method for actuating a pump for delivery of a fluid. In a specific embodiment, the fluid is a drug. The method distinguishes an actuation mechanism for the pump. This actuation mechanism has a tubular structure with a first end and a second end, and an outer surface. The outer surface has helical grooves over a length in between the first end and the second end of the outer surface. A shape memory alloy coiled-wire is positioned over the length and in the helical grooves. The position or placement allows for a space between the shape memory alloy coiled-wire and helical grooves for the shape memory alloy coiled-wire to twist freely. In some examples, the number of turns, loops or revolutions of the coil can be at least 1, or at least 2 or at least 3. The actuation mechanism further distinguishes an enclosure fitting encompassing the tubular structure therewith the shape memory alloy coiled-wire and the helical grooves. The method then further distinguishes placing a fluid inside the tubular structure. The shape memory alloy coiled-wire can be activated, which causes a shortening of the tubular structure over the length in between the first end and the second end and therewith a compression of the fluid resulting in a release of a fluid from the tubular structure. Deactivation or stopping of the activation causes the shortening of the tubular structure to be reversed for the tubular structure; the reversal is caused by a stiffness of the enclosure.

In a further embodiment, the actuation mechanism further includes a fluid-reservoir such that the method further includes placing the fluid-filled reservoir inside the tubular structure. The shortening causes a compression of the fluid-reservoir and therewith a release of the fluid from the fluid-reservoir.

In one embodiment the released fluid can be captured in an in between space in between an inner valve and an outer valve (i.e. a temporary holding space) to then be released when the reversal of length occurs.

In still another embodiment, the enclosure has a self-healing membrane at either the first or the second end, such that the method further includes the step of filling the tubular structure with a fluid by puncturing a needle through the membrane and injecting the fluid inside the tubular structure.

The invention is further captured by the device of the pump and its structural components as described herein.

The main advantage of the device over the existing art is the pure linear actuation of the SMA wires which results in large linear deflections and high force, which then translates to increased throughput and high output pressure. Moreover, the pure quasi-static linear motion (i.e. avoiding buckling and structural instability) allows better control over the flow rates. Finally, the replaceable reservoir facilitates pump reuse. The above features make the pump better optimized for drug delivery applications.

### BRIEF DESCRIPTION OF THE DRAWINGS

FIG. 1A shows according to an exemplary embodiment of the invention force-deflection curves for the shape memory alloy (SMA) wire in its 100% austenite and 100% martensite phase, and the elastomeric enclosure in relation to them.

FIG. 1B shows according to an exemplary embodiment of the invention SMA's crystalline arrangement during the different operation stages.

FIG. 2 shows according to an exemplary embodiment of the invention in a 3D-printed valve cross-section. The figure as shown has the valve in closed mode, but a skilled artisan would appreciate what the open mode looks like.

FIGS. 3A-D show according to an exemplary embodiment of the invention the maximum deflection (in mm) as a function of the wire diameter (x-axis, in mm) and the number of turns (y-axis). The regions inside the black boundary represent the designs that cause the SMA coil to deform plastically, whereas the regions outside the black boundary represent the elastic deflections. The red trend lines follow the peak points at which there is maximum displacement for the wire thickness values. Enclosure material curves for (FIG. 3A) Ecoflex 00-10 (FIG. 3B) Ecoflex 00-30 (FIG. 3C) Ecoflex 00-50 (FIG. 3D) PDMS.

FIGS. 4A-G show according to an exemplary embodiment of the invention a process flow diagram and exploded views of the pump and pump mechanisms. For reference item numbers please refer to Table 1.

FIG. 5 shows according to an exemplary embodiment of the invention pump discharge and input power vs. number of SMA wire turns.

FIGS. 6A-B show according to an exemplary embodiment of the invention force-displacement curve of the SMA wire in austenite and martensite phases, and the Ecoflex enclosure for (FIG. 6A) 3-turn and (FIG. 6B) 6-turn pumps, at wire diameter=0.4 mm. The pump's full range of motion from martensite to austenite is represented by δ.

FIGS. 7A-B show according to an exemplary embodiment of the invention volume flow rate vs. frequency at 2.5, 3.5, and 5 V for the 3-turn pump at (FIG. 7A) duty cycle 1:1 (FIG. 7B) duty Cycle 1:2. FIG. 7A also shows forced convection cooling mode at 5 V.

FIGS. 8A-B show according to an exemplary embodiment of the invention in FIG. 8A pump displacement vs. time at duty cycles of 1:1 and 1:2 and forced convection conditions. In FIG. 8B speed vs. input current for the 3- and 4-turn pumps. Cycle time=4 s, for the 1:1 cycle, T=2 s, and 6=2 s. For the 1:2 cycle, T=1.33 s, and 6=2.66 s.

FIGS. 9A-B show according to an exemplary embodiment of the invention in FIG. 9A pump's volume flow rate and input power vs. SMA wire diameter. In FIG. 9B pump discharge volume at different SMA wire diameters.

FIGS. 10A-C show according to an exemplary embodiment of the invention force-displacement curve of the SMA in austenite and martensite phases, and the Ecoflex enclosure for SMA wire diameters of (FIG. 10A) 0.25, (FIG. 10B) 0.4, and (FIG. 10C) 0.5 mm for the 8-turn devices. The pump's full range of motion from martensite to austenite is represented by S.

FIG. 11 shows according to an exemplary embodiment of the invention pump's static head pressure for the 3, 4, 6, and 8-turn pumps.

FIG. 12 shows according to an exemplary embodiment of the invention discharge volume, the static head pressure, and the current profile for the 3-turn pump operating at 790 mA input current in a controlled pump operation mode.

FIG. 13 shows according to an exemplary embodiment of the invention a tubular capsule with a self-healing membrane at one end. The self-healing membrane can be punctured with a needle to fill the tubular structure with a fluid by injecting the fluid inside the tubular structure.

### DETAILED DESCRIPTION

Unlike the typical diaphragm and peristaltic designs, embodiments according to this invention of a high-power density resistively actuated SMA capsule micropump for drug delivery applications are actuated linearly in the most compliant direction allowing large displacements, large discharge volume, and high throughput. The pump's capsule design integrates a replaceable drug reservoir that can be preloaded with the desired drug within the pump package, increasing the fill-factor and reducing the overall footprint. The overall area occupied by the pump, including the actuation mechanisms, the valves, and the drug reservoir. The pump integrates all these components in one self-contained package, thus reducing the overall occupied area. In one example, the pump structure is made out of an elastic rubber material that acts as a spring and is useful in recovering from the contraction after the actuation (i.e. from the detwinned to the twinned phase). The structure is also flexible, insulated, and biocompatible for drug delivery. The reservoir is replaceable, which means that the pump can be reusable many times and for different drug types. This makes the whole package compact, efficient, cheap, and compatible for drug delivery applications.

Pump Characteristics

The pump concept is based on winding SMA wires on a flexible elastomeric capsule. Upon heating the spring, SMA converts the heat energy into a linear displacement that leads to injecting the drug through a one-way valve to the desired tissue. Upon running off the current, the detwinning is obtained by the polymer capsule's stiffness, thus resetting the pump to the initial position. In one example, NiTi SMA wires were selected to increase the heat transfer rate compared to the polymer enclosure. The NiTi SMA wires with a round cross-section measuring 0.25, 0.4, and 0.5 mm in diameter were coiled on 6-mm diameter elastomeric enclosure (i.e. capsule) spring diameter (i.e., 21.2 mm² cross-sectional area, 424.7 mm³ volume). The actuation frequency of the SMA spring is dependent on the pulse of the voltage and the current from the supply. Each pulse's on-time results in the pump's contraction, whereas during off-time, the pump recovers its position and takes up the drug from the integrated reservoir.

Pump Operation

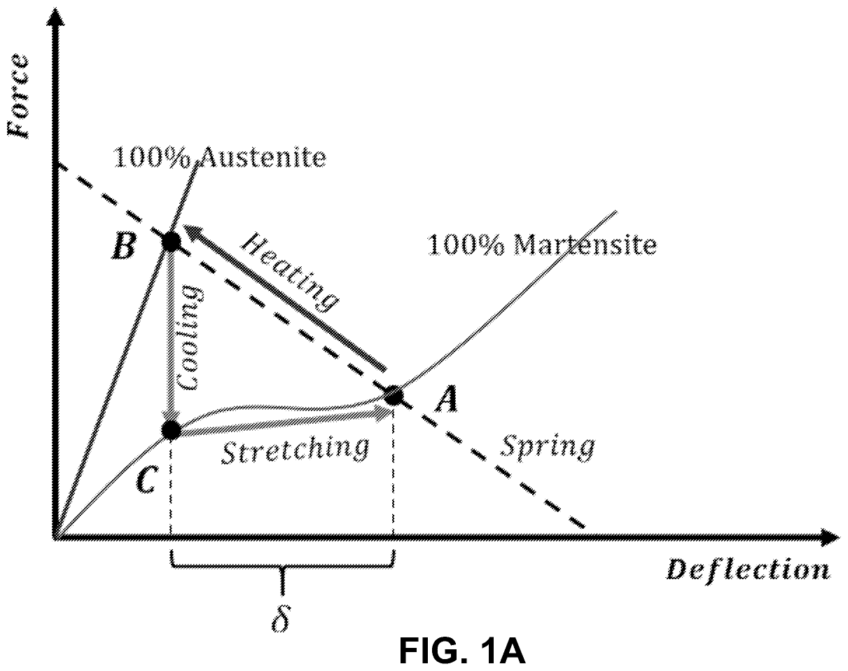

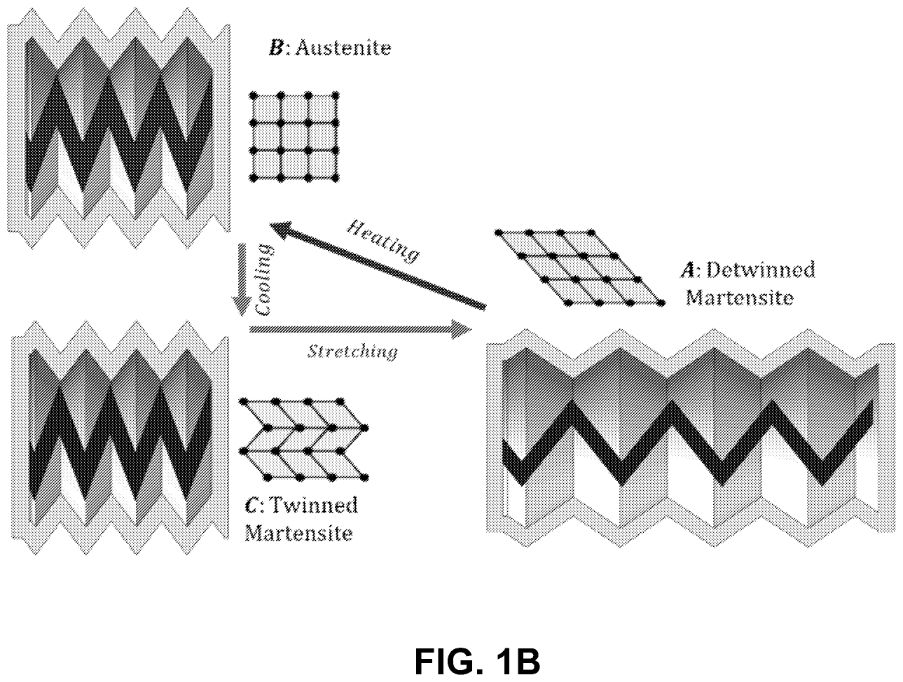

The SMA wire (1) can be compressed through resistive heating. However, to stretch the SMA wire back to its stretched form, an opposing element must be added to apply a restoring force. In one design, the restoring element is the elastomeric enclosure (2). At any point in the operation, the pump's instantaneous deflection is the point at which the coil and elastomeric enclosure apply the same force (FIG. 1A). Since the SMA coil's force-deflection behavior changes with the instantaneous SMA material phase, the intersection points to move, causing a deflection in the pump.

When the current passes through the SMA wire, its temperature will rise by the joule heating effect. This causes a phase change in the SMA spring to the austinite phase. The force-deflection curve of the SMA spring in the austenite phase is significantly larger than that of the martensite. This causes the spring to contract along with the elastomeric enclosure. This deflection causes fluid to be pumped out equal to the change in volume. After this contraction, the current is turned off and left to cool down. As it cools down, SMA will attempt to restore back to the twinned martensite phase. This phase has a lower force-deflection curve, so the stiffness of the deformed elastomeric enclosure will recover and stretch the coil back to its initial position. As the coil is stretching, it gradually changes the inner structure to detwinned martensite (FIG. 1B). During this phase, fluid is pulled into the pump from the built-in drug reservoir and ready to pump the drug in the next cycle. The maximum fluid pressure pumped during compression is caused by the difference in force between the SMA coil in its austenite phase and the elastomeric enclosure. During the expansion phase, the pump's negative pressure pulls fluid in from the reservoir with force applied by the elastomeric enclosure.

Moreover, to achieve pure linear motion, avoid structural instability, and allow better control over the pressure and discharged volume, the SMA wire must be allowed to twist freely within the enclosure (i.e. without transmitting the twisting motion to the elastomers). This design achieves this by making a clearance 9 around the SMA wire.

Design

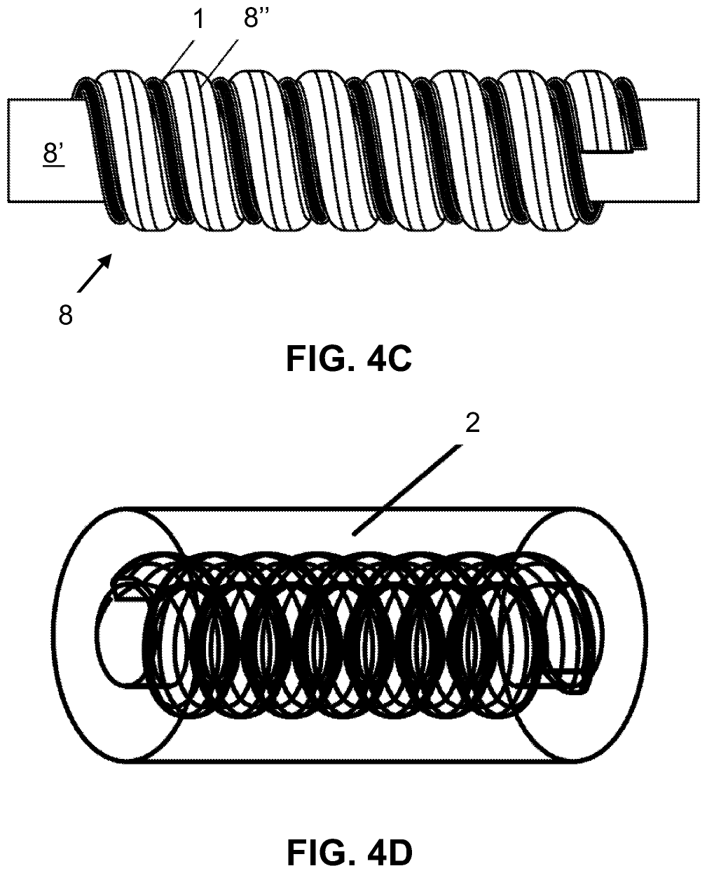

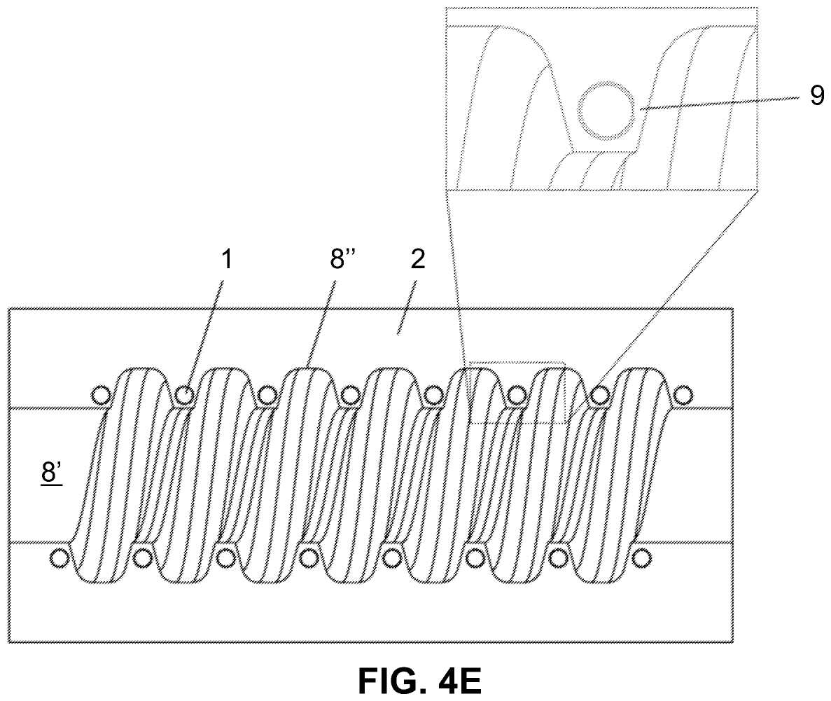

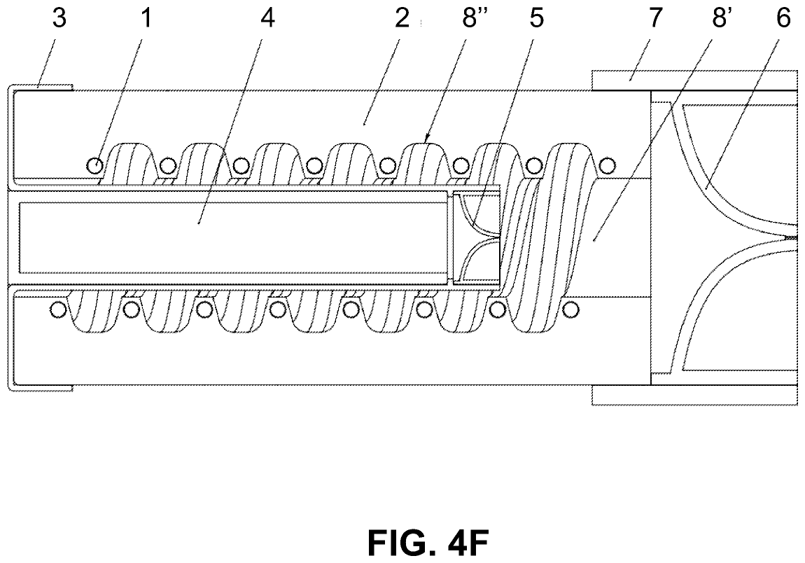

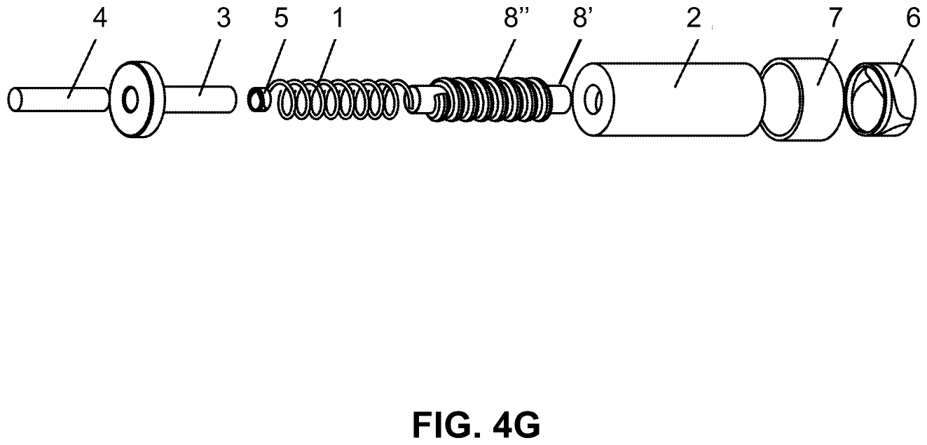

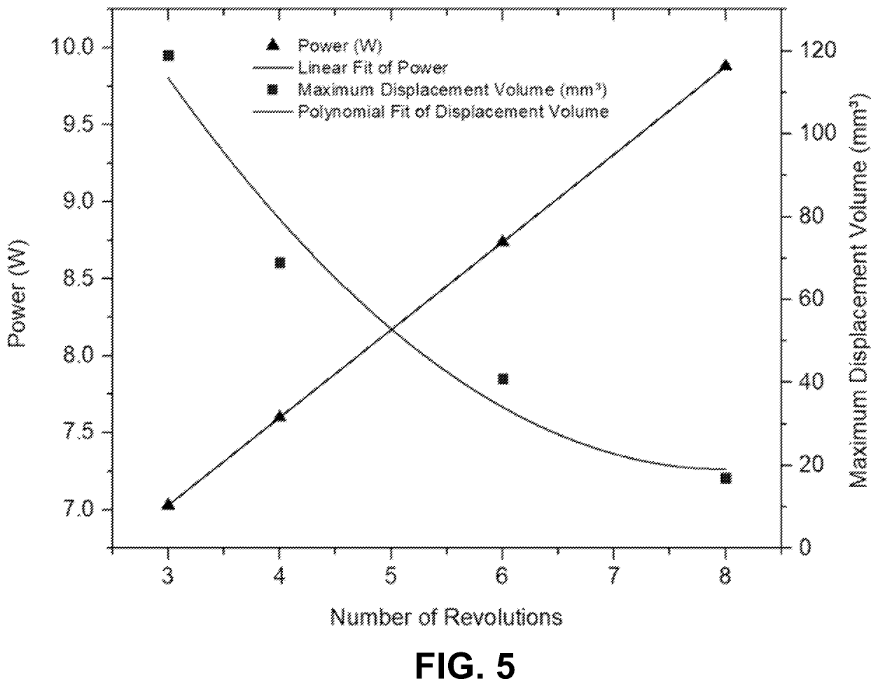

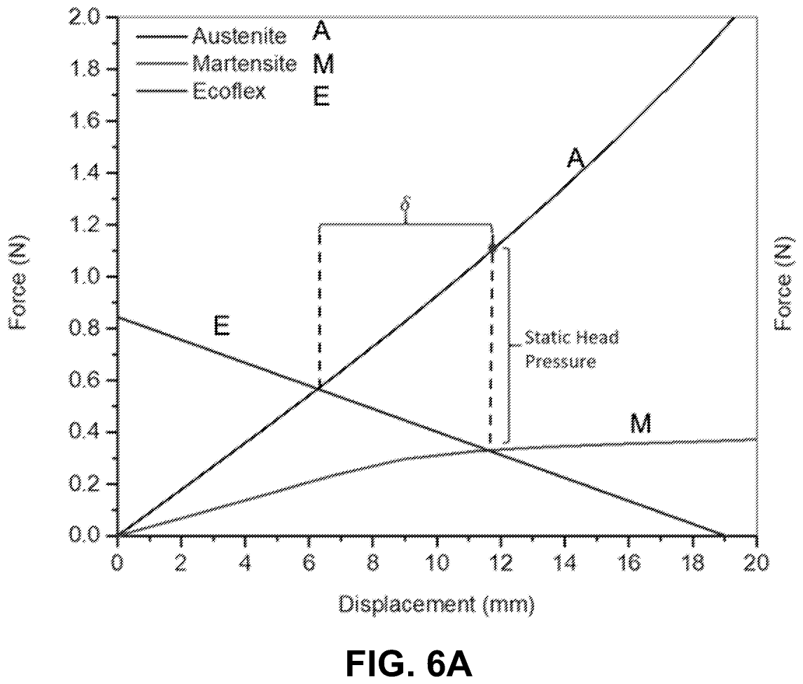

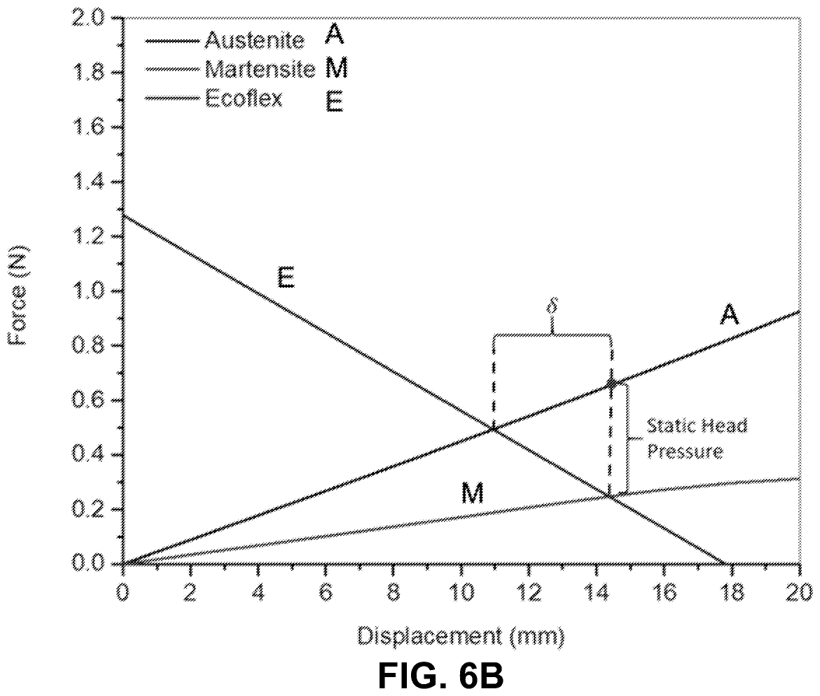

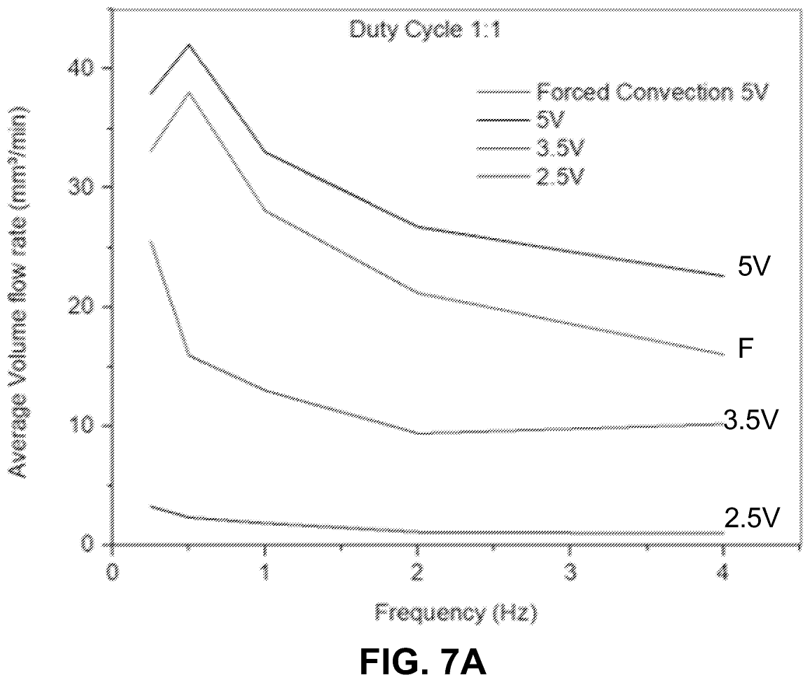

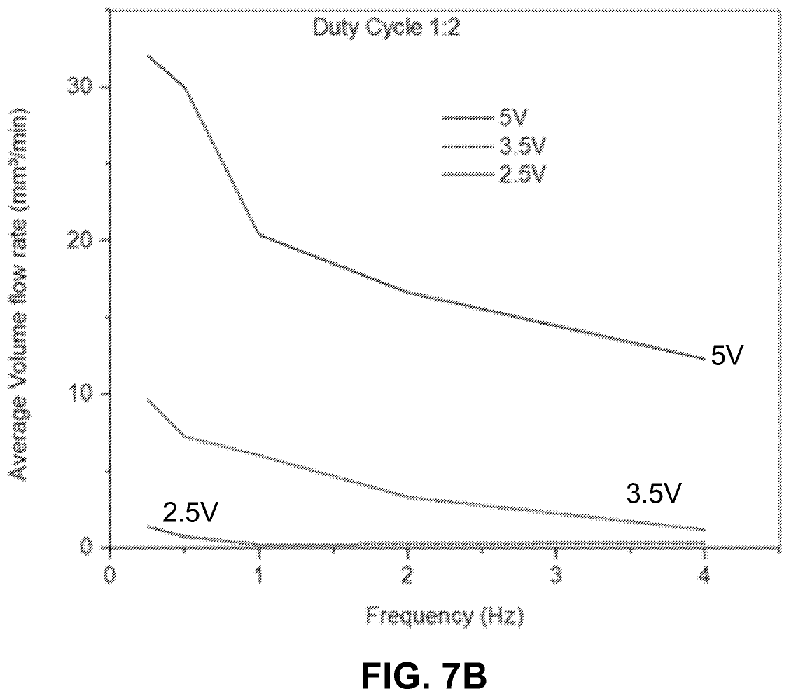

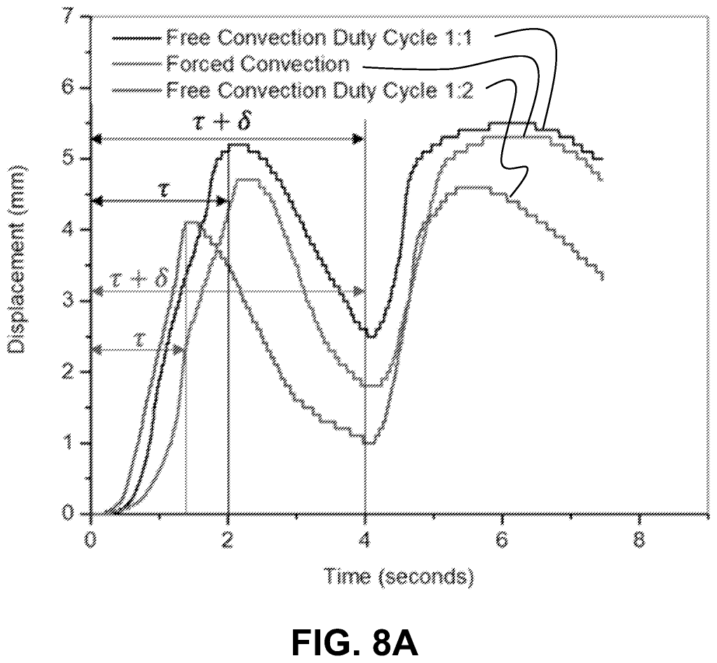

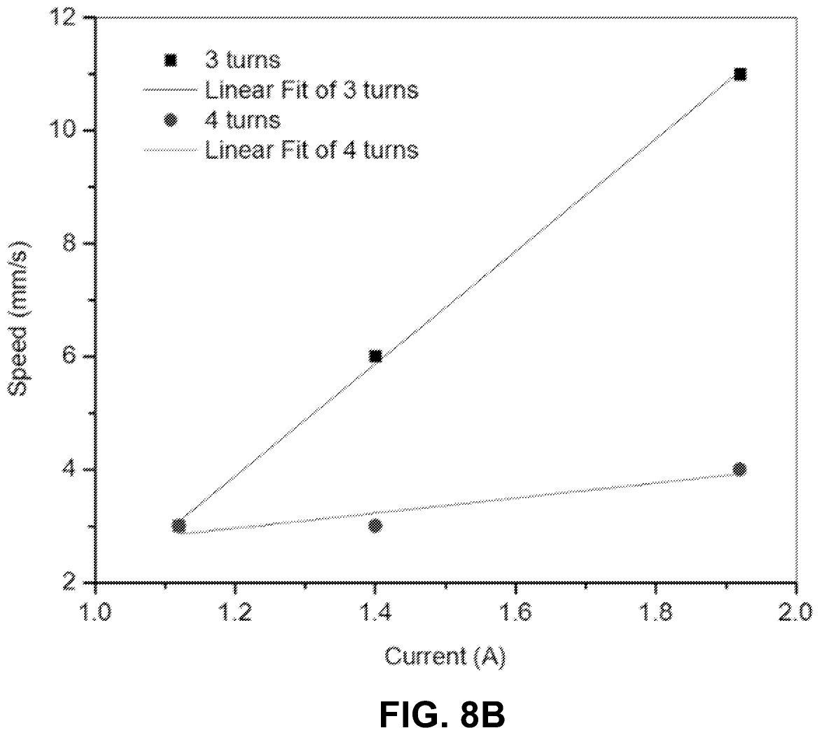

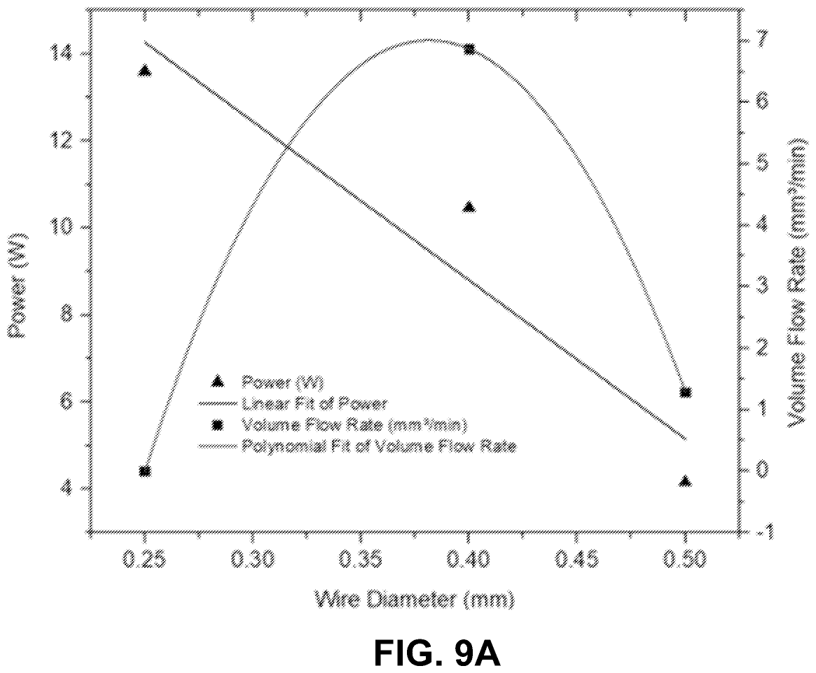

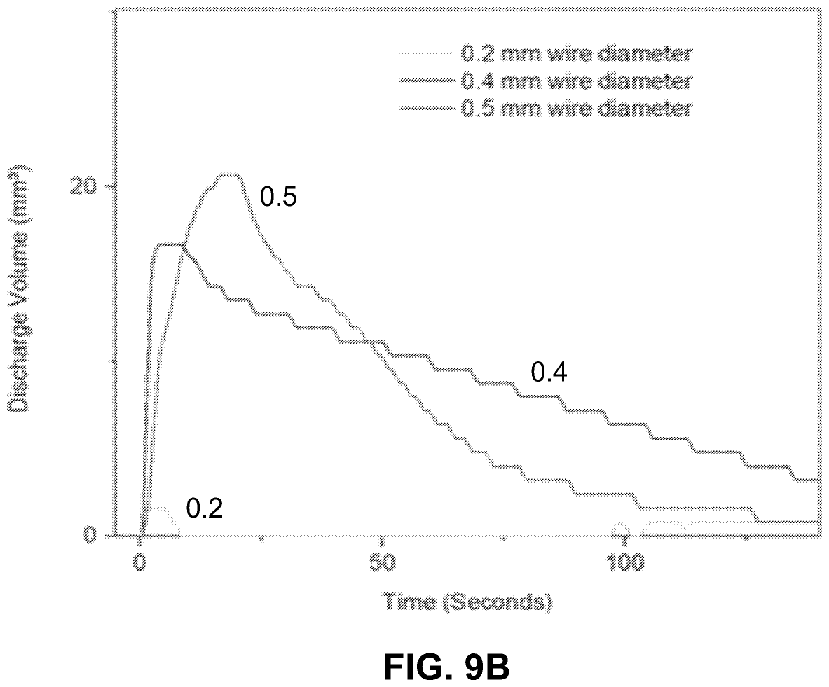

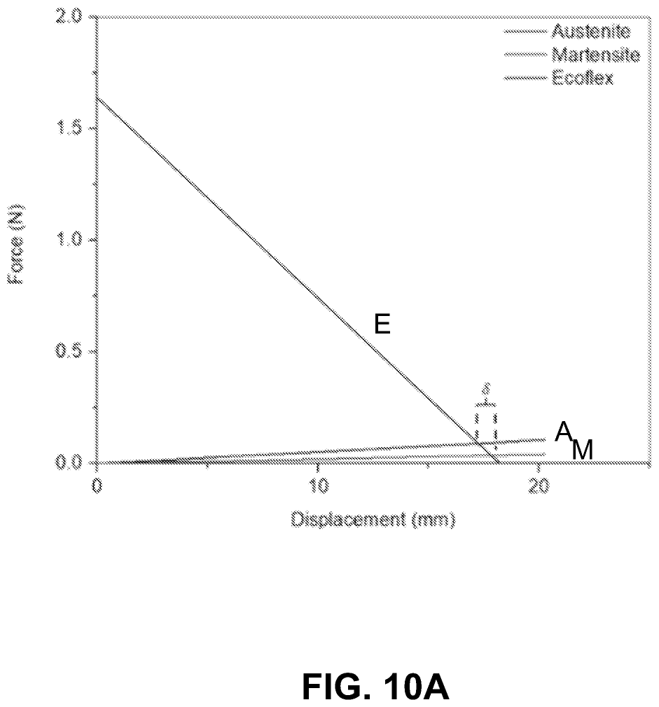

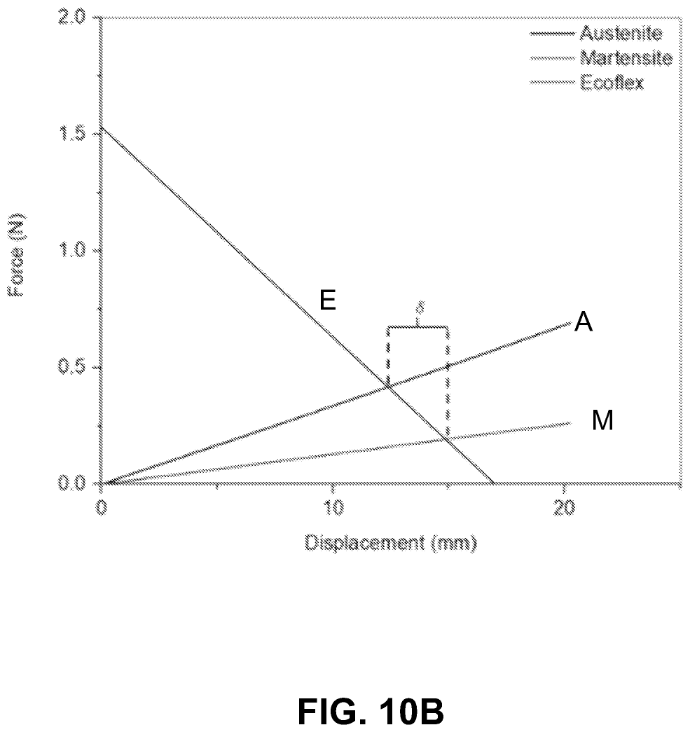

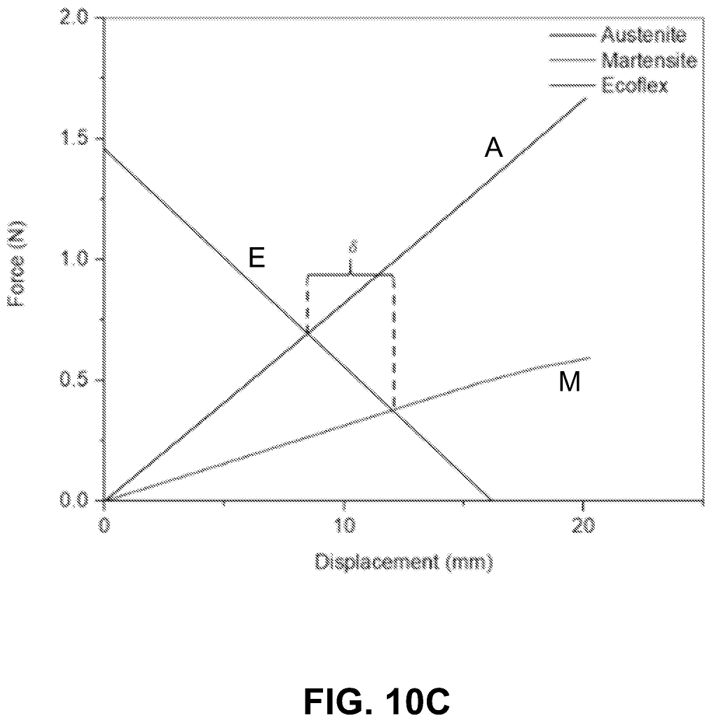

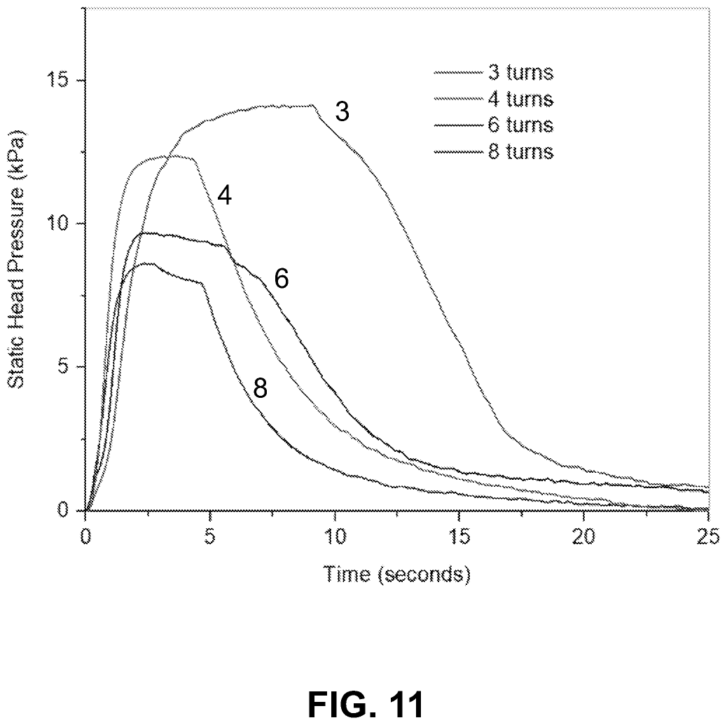

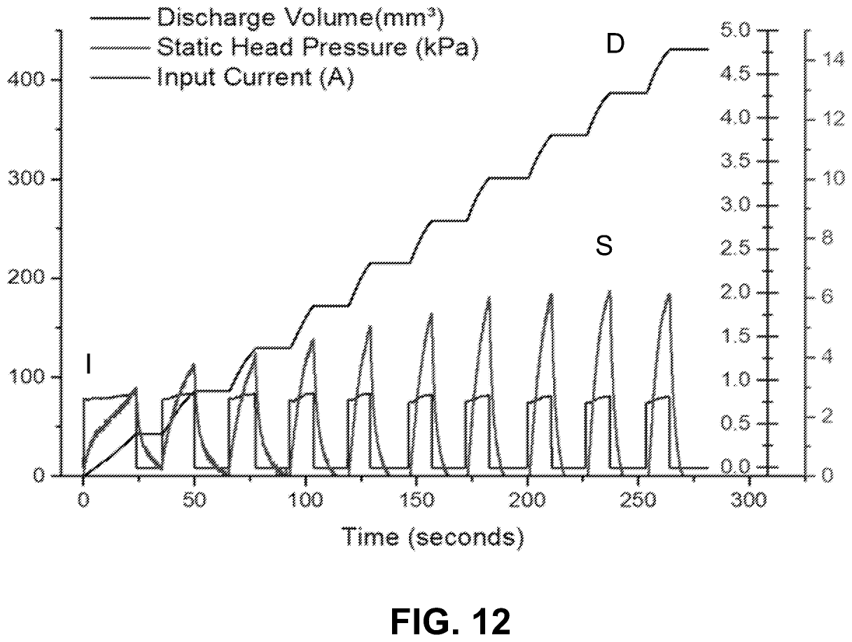

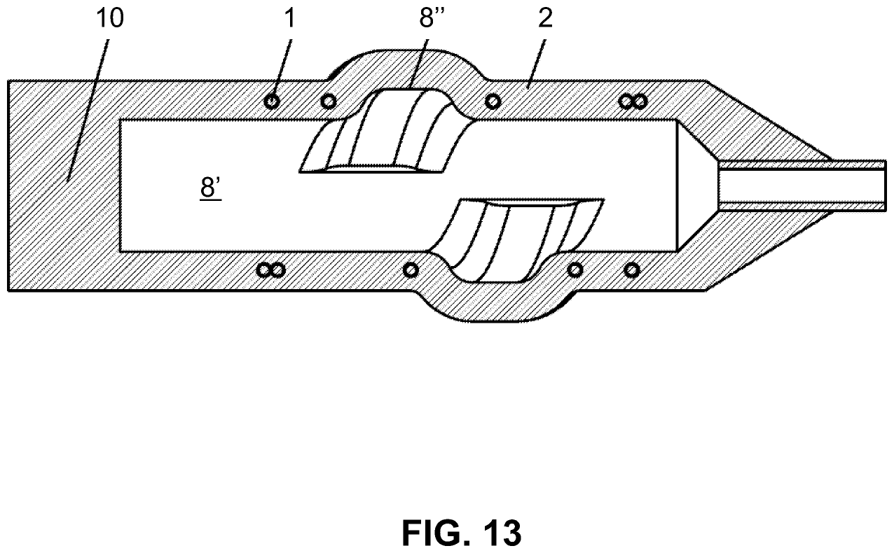

In the embodiments of the design, referring to FIGS. 4A-G, the twisting degree of freedom can be canceled out and generate a purely linear motion over a certain length, which is significant in the pump's performance and control. The design integrates a built-in replaceable drug reservoir 4 within the pump package forming a self-contained preloaded capsule pump. The design has space/clearance 9 for the SMA wire 1 to twist freely, thus relaxing its twist off the rubber capsule. The pump has NiTi-alloy SMA wires 1 coiled on an elastomeric capsule 8 with helical grooves/ridges 8′ and actuated by Joule heating (i.e., by passing electric current). When the SMA wire is actuated, it allows wire twist motion to generate freely; the wire tries to get shorter, pushing the ridges within capsule 8 together and causing a linear motion (i.e. linear stroke), pushing out fluid from the pump through the outer valve 6. Then after this process, the SMA wires are relaxed (deformed) back to the initial position by the stiffness of the elastomeric enclosure 2, allowing the fluid to come up from the replaceable reservoir to the inside of the pump. A sleeve 3 contains the replaceable reservoir, and the inner valve 5 connects the reservoir to the main pump chamber. It also functions as fixed support to the pump. It fixes one end of the pump for the SMA wire to contract, thus pushing out the pump's volume without squeezing the replaceable reservoir.

TABLE 1
Design Items corresponding to FIGS. 4A-F.

| Item # | Item Description |
|---|---|
| 1 | SMA Wire |
| 2 | Elastomeric Enclosure |
| 3 | Sleeve |
| 4 | Replaceable Reservoir |
| 5 | Inner Valve |
| 6 | Outer Valve |
| 7 | Valve Holder |
| 8, 8′, 8″ | Elastomeric capsule 8 distinguishing tubular |
| | structure 8′ and on outer surface helical |
| | grooves 8″ |
| 9 | Space between the shape memory alloy |
| | coiled-wire and helical grooves for the shape |
| | memory alloy coiled-wire to allow it to twist |
| | freely |
| 10 | Self-healing puncturable membrane |

Valve Design

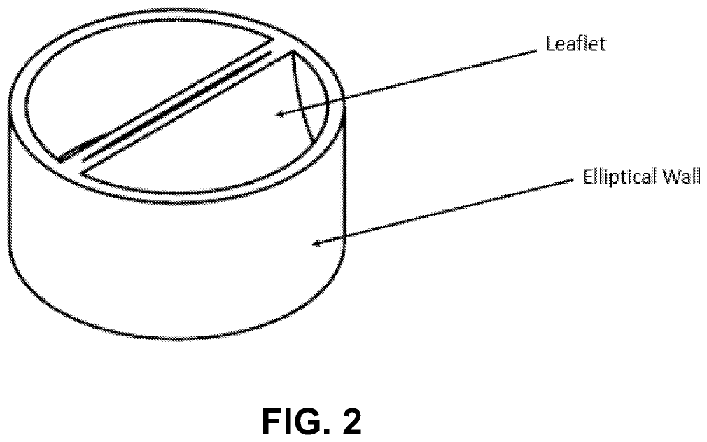

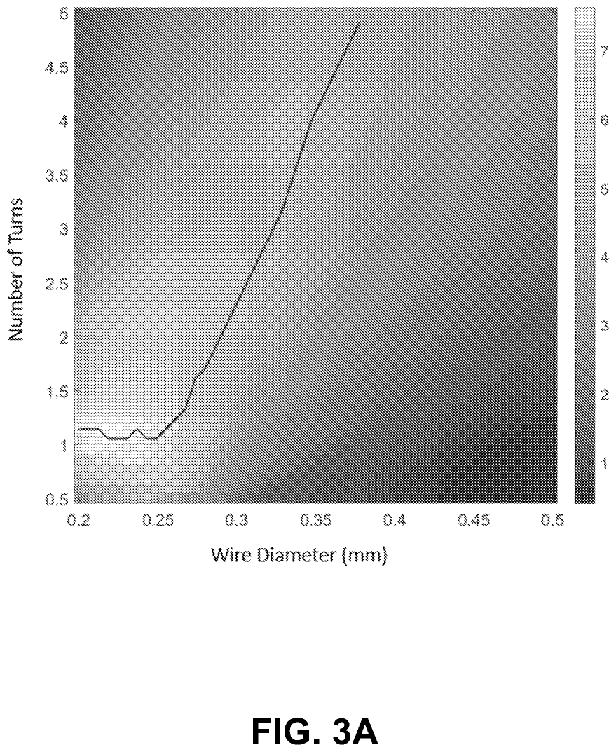

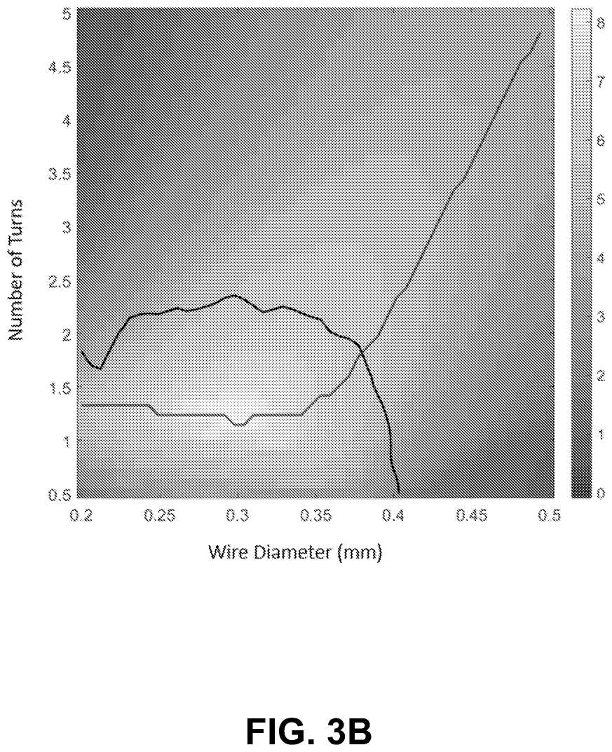

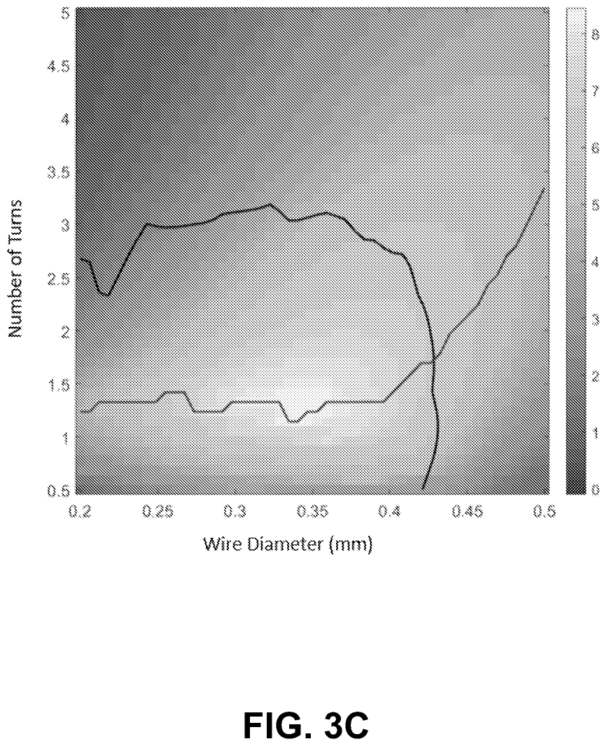

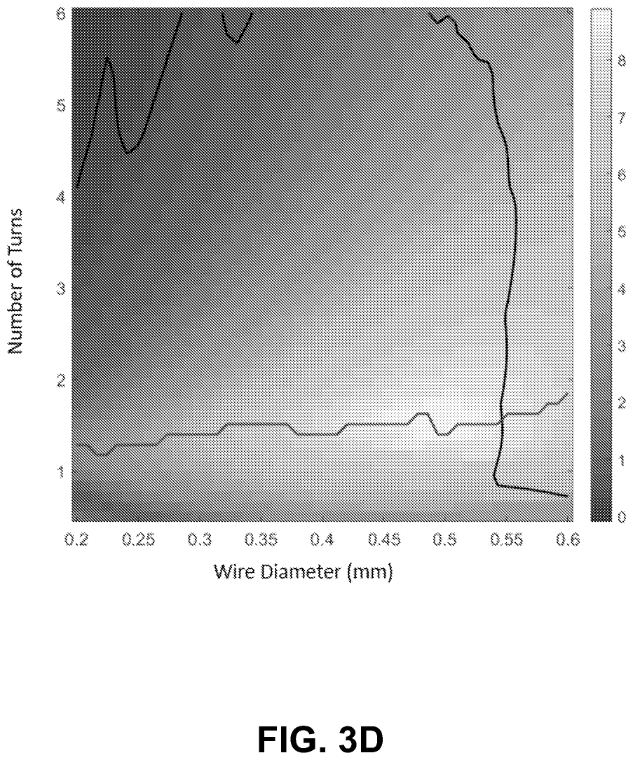

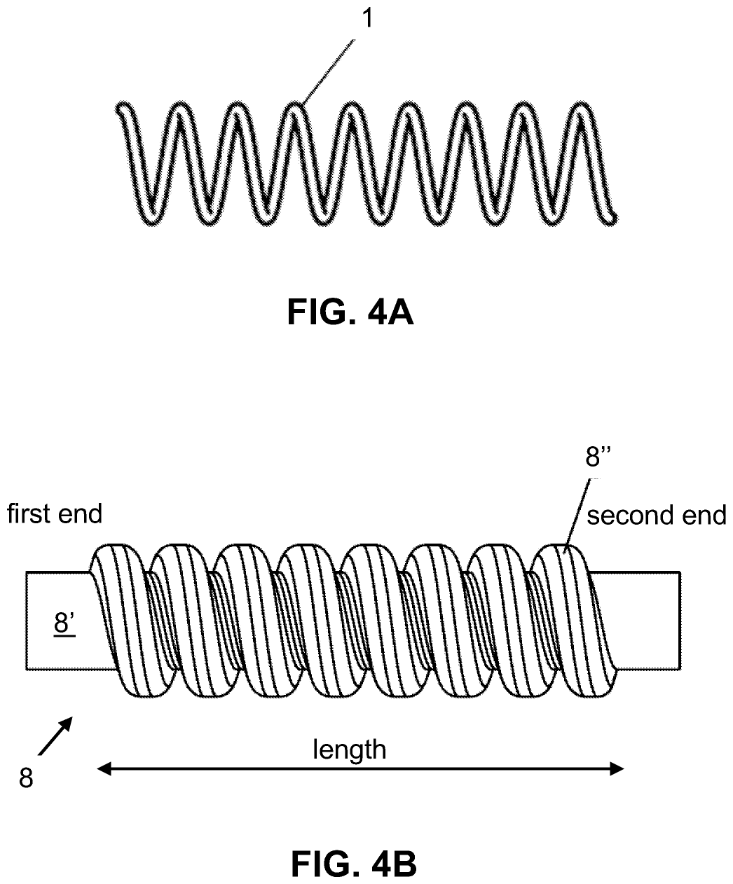

The valve's design is inspired by the bicuspid aortic valve. It is composed of two flexible leaflets opposing each other connected to a flexible cylindrical wall (FIG. 2). To ensure that the valve is fully closed in the absence of fluid flow, the leaflets are spatially constrained to push against each other. The valve's elliptical shape makes it preloaded such that it is compressed in the direction normal to the leaflets and constraining any leakage. The leaflets are curved in such a way to allow internal pressure to push the leaflets apart, and external fluid pressure pushes the two leaflets together during the pumping cycle. The valve was printed as a single part by stereolithography 3D printing with desired stiffness using Formlab's Shore 80 A flexible resin.

Materials

According to an exemplary embodiment, NiTi wires of diameters 0.25, 0.4, and 0.5 mm with the transition temperature of 45° C. were obtained from Kellogg's Research Lab, USA. Designing an SMA micropump requires a flexible structural material enclosure material with the correct stiffness to restore the SMA phase. Moreover, the enclosure material must be easily molded and cured at a temperature lower than the austenite transition temperature. Also, based on the above-mentioned model results, it has to have a small young's modulus to minimize the SMA wire size and power consumption. Therefore, Ecoflex 00-30 platinum silicone was chosen for its convenient mechanical properties, namely its large elongation, ease of molding due to low mixed viscosity, short room temperature curing time (Table 2) and biocompatibility. It was chosen over Ecoflex 00-10 grade due to its strength and mechanical robustness, which will allow the pump to work over larger number of cycles. Functionally, Ecoflex 00-10 is −4.7 times more viscous than 00-30 (rated at 14 Pa*s), which compromises the molding of the fine pump features. Moreover, Ecoflex 00-10 has 40% less tensile strength than 00-30 (rated at 0.83 MPa) and 42% less tear strength (3.85 N/mm versus 6.65 N/mm). This will affect the device's reliability since the pump's operation requires stretching (i.e., elongation >27%) the polymer many times and rebounding to the original position, which increases the possibility of tearing at the 00-10 lower tear and tensile strengths.

Ecoflex 00-30 was also chosen over PDMS based on the model results to allow the usage of a thinner wire diameter and reduce the current and power consumption.

TABLE 2
Ecoflex 00-30 material properties.

| Material Property | Value |
|---|---|
| Mixed Viscosity [Pa*s] | 3 |
| Specific Gravity [g/cm³] | 1.07 |
| Shore Hardness [ASTM D-2240] | 00-30 |
| Tear Strength [N/mm] | 6.65 |
| Tensile Strength [MPa] | 1.38 |
| Elongation at break [%] | 900 |
| Shrinkage [mm/mm] | <0.001 |
| Curing Time at Room Temperature [h] | 4 |

Pump Fabrication

A four-step process was optimized to fabricate the SMA micropumps, as shown in FIGS. 2A-G. Ecoflex (i.e., elastomer) was shaped in a 3D printed mold to form the inner pump structure. In the next step, the SMA wires were coiled along the inner structure's helical grooves with the desired number of turns (1, 2, 3, 4, 6, and 8 turns). Next, the outer capsule is shaped in a 3D printed mold and assembled with the inner structure. The drug reservoir is shaped using Ecoflex 00-30 in one step in an aluminum mold. The inner and the outer valves are each printed as a single part through Stereolithography with flexible 80 A resin by Formlabs, USA. A sleeve 3 was used for containing the replaceable reservoir and the inner valve that connect the reservoir to the main pump chamber. It also functions as a fixed support to the pump as it fixes one end of the pump for the SMA spring to contract, thus pushing out the pump's volume without squeezing the reservoir. Finally, the pump structure, the sleeve, the reservoir, and the valves are assembled, as shown in FIG. 4G show schematics of the pump assembly and actual picture of the developed pump components, respectively.

Characterization

The pump's displacement, discharge, volume flow rate, static pressure head, and the Ecoflex's and SMA's force-displacement responses were experimentally characterized using force (resolution=0.03 N) and linear motion (resolution=0.0078 mm) sensors by PASCO, USA.

What is claimed is:

1. A method for actuating a pump, comprising:
(a) having an actuation mechanism for the pump, wherein the actuation mechanism comprising:
(i) a tubular structure with a first end and a second end, and with an outer surface, wherein the outer surface has helical grooves over a length in between the first end and the second end of the outer surface,
(ii) a shape memory alloy coiled-wire positioned over the length and in the helical grooves, wherein the position allows for a space between the shape memory alloy coiled-wire and helical grooves for the shape memory alloy coiled-wire to twist freely; and
(iii) an enclosure fitting encompassing the tubular structure therewith the shape memory alloy coiled-wire and the helical grooves;
(b) placing a fluid inside the tubular structure; and
(c) activating the shape memory alloy coiled-wire which causes a shortening of the tubular structure over the length in between the first end and the second end and therewith a compression of the fluid resulting in a release of a fluid from the tubular structure.

2. The method as set forth in claim 1, wherein the shape memory alloy coiled-wire has at least one loop or revolution.

3. The method as set forth in claim 1, wherein the shape memory alloy coiled-wire has at least three one loops or revolutions.

4. The method as set forth in claim 1, wherein the shortening of the tubular structure is followed by a reversal of the shortening of the tubular structure when the actuation has stopped, wherein the reversal is caused by a stiffness of the enclosure.

5. The method as set forth in claim 1, wherein the actuation mechanism further comprises a fluid-reservoir and wherein the method further comprises placing the fluid-filled reservoir inside the tubular structure, wherein the shortening causes a compression of the fluid-reservoir and therewith a release of the fluid from the fluid-reservoir.

6. The method as set forth in claim 1, wherein the enclosure comprises a self-healing membrane at either the first or the second end, and wherein the method further comprises filling the tubular structure with a fluid by puncturing a needle through the membrane and injecting the fluid inside the tubular structure.
